(19) United States
(12) Patent Application Publication
(10) Pub. No.: US 2019/0072563 A1
(43) Pub. Date: Mar. 7, 2019

(54) METHOD FOR EARLY DIAGNOSING AND TREATING ACUTE CORONARY SYNDROME

(71) Applicant: Chi-Hua Foundation, New Taipei City (TW)

(72) Inventor: WAN-LIN WU, New Taipei City (TW)

(21) Appl. No.: 15/697,603

(22) Filed: Sep. 7, 2017

(51) Int. Cl. G01N 33/68

(52) U.S. Cl. CPC G01N 33/68; G01N 2800/324; A61B 5/02; G01N 2333/47; G01N 2800/60

(57) ABSTRACT

A method for early diagnosis and treatment of acute coronary syndrome (ACS) in a subject, which measures a level of at least one specific circulating biomarker using immunomagnetic reduction, e.g. FABP4, in the subject within a certain time period after onset of at least one symptom of ACS and administers guideline recommended therapies including early reperfusion therapy and effective drugs to the subject who is observed to have significant dynamic changes in the level of the at least one specific biomarker.

### BACKGROUND OF THE INVENTION

### Field of the Invention

The present invention relates to a method for early diagnosis of acute coronary syndrome (ACS) in a subject by detecting a concentration of at least one cardiac marker.

### Description of Prior Art

Cardiovascular diseases (CVD) represent the leading cause of mortality worldwide, despite the remarkable advances in interventional cardiology, cardiac surgery, and modern pharmacotherapy, particularly in the setting of acute myocardial infarction (AMI), chronic ischemic heart failure (HF), cardiomyopathy (CM), or the associated left ventricular (LV) dysfunction. CVD is a group of diseases that involve the myocardium, valves, and blood vessels, thereby including coronary artery disease (CAD) and several other conditions. Individuals with CAD can be grouped into individuals who do not exhibit chest pain, and those who exhibit chest pain. The latter group can be grouped into individuals who exhibit a stable angina pectoris (SAP), and those who with ACS. On the other hand, individuals presenting with undifferentiated chest pain could be grouped into ACS or non-ACS.

ACS refers to a spectrum of clinical presentations ranging from those for ST-segment elevation myocardial infarction (STEMI) to presentations found in non-ST-segment elevation myocardial infarction (NSTEMI) or in unstable angina pectoris (UAP). It is almost always associated with rupture of an atherosclerotic plaque and partial or complete thrombosis of the infarct-related artery. ACS patients can exhibit UAP, or these patients already suffered from AMI. The AMI can be an STEMI or a NSTEMI. The occurrence of an AMI can be associated with a LVD. LVD patients may experience symptoms of HF with a mortality rate of about 15%.

Due to acute chest pain is a frequent symptom in medical emergency departments, distinguishing patients with ACS within the chest pain group is a diagnostic challenge. At present, ACS is diagnosed on the basis of typical angina symptom, electrocardiogram (ECG) results and serial cardiac enzymes analysis, among which the serial cardiac enzymes measurements play a vital role in the diagnosis of AMI. Patients with two positives out of three clinical criteria are diagnosed with ACS. The cardiac enzymes (e.g. creatine phosphokinase (CPK)/CK-MB, troponins and myoglobin) are released after myocardial cell disintegration and are markers of cell necrosis. However, the rise time and metabolic cycle of each cardiac enzyme after myocardial injury are not the same and the sensitivity and specificity of each cardiac enzyme are different, it may produces false negativity when only one of these cardiac enzymes is detected at a certain point. Therefore, it needs continuously and repeatedly detect the metabolic cycle of one or more cardiac enzymes for clinical diagnosis of ACS. The process of this detecting method takes too long to make rapid diagnoses.

A rapid diagnosis of ACS is a clinical and operational priority in emergency departments. Serial serum levels of cardiac biomarkers are heavily involved in the evaluation of patients presenting with acute chest pain, so an accurate and rapidly-responsive assay of cardiac biomarkers plays a critical role at emergency departments. Thus, it is desirable to have an effective detecting method and novel biomarkers which provide accurate and rapid diagnoses of ACS.

### SUMMARY OF THE INVENTION

Blood-borne biomarkers reflecting atherosclerotic plaque burden have great potential to improve clinical management of atherosclerotic CAD and ACS. Immunomagnetic reduction (IMR) has been developed for rapid and on-site assay with small sample volume. The reagent is a solution of homogeneously dispersed magnetic nanoparticles, which are coated with hydrophilic surfactants and bioprobes. When magnetic nanoparticles bind with the bio-probes on the outermost shell and become larger or clustered, the concentration can be measured quantitatively. The present invention develops IMR kits for multiple biomarkers (myoglobin [MyG], creatine kinase-MB [CK-MB], troponin-I [Tn-I]), and fatty acid-binding protein 4 [FABP4]).

Thus, the present invention provides a method for early diagnosis and treatment of ACS in a subject, comprising measuring a level of at least one specific serum biomarker in the subject within a certain time period, often 72 hours after onset of acute chest pain, and administering effective therapies including reperfusion therapy (e.g. thrombolytic therapy, coronary angioplasty or bypass surgery, etc.) and medications to the subject who is observed to have significant dynamic changes in the level of the at least one specific biomarker.

### BRIEF DESCRIPTION OF THE DRAWINGS

FIG. 1 shows the concentration-dependent immunomagnetic reduction (IMR) signal curve with error bars for each cardiac marker. (A) shows the concentration-dependent IMR signal curve of myoglobin. (B) shows the concentration-dependent IMR signal curve of CK-MB. (C) shows the concentration-dependent IMR signal curve of Tn-I. (D) shows the concentration-dependent IMR signal curve of FABP4.

FIG. 2 shows myoglobin (MyG) time activity curves in patients with STEMI who undergo primary coronary intervention (n=6). The peaking hour of MyG is 8.2 hr. Fit: fitting curve.

FIG. 3 shows creatine kinase-MB (CK-MB) time activity curves in patients with STEMI who undergo primary coronary intervention (n=6). The peaking hour of CK-MB is 24.4 hr. Fit: fitting curve.

FIG. 4 shows troponin-I (Tn-I) time activity curves in patients with STEMI who undergo primary coronary intervention (n=6). The peaking hour of Tn-I is 24.7 hr. Fit: fitting curve.

FIG. 5 shows fatty acid-binding protein 4 (FABP4) time activity curve in patients with STEMI who undergo primary coronary intervention (n=6). The peaking hour of FABP4 is 10.5 hr. Fit: fitting curve.

### DETAILED DESCRIPTION OF THE INVENTION

The present invention demonstrates the relationship between the acute coronary syndrome (ACS) and the concentrations of specific circulating biomarkers. The significant dynamic changes in the concentrations of the circulating biomarkers within certain time period after onset of acute chest pain in a patient can be used to diagnose whether the patient has ACS. The present invention allows medical professionals to early diagnose whether a patient is suffering from ACS by a fast and easy detecting method.

As used herein, “a” or “an” may mean one or more. As used herein in the claim(s), when used in conjunction with the word “comprising”, the words “a” or “an” may mean one or more than one.

The present invention provides a method for early diagnosing and treating acute coronary syndrome (ACS) in a subject in need thereof, the method comprising: (a) providing a first blood sample collected at a first time point from the subject, wherein the first time point is within 120 hours after onset of at least one symptom of ACS in the subject; (b) determining a first concentration of at least one cardiac marker in the first blood sample collected at the first time point by performing an immunomagnetic reduction (IMR) assay, wherein the at least one cardiac marker comprises a fatty acid-binding protein 4 (FABP4); (c) providing a second blood sample collected at a second time point from the subject, wherein the second time point is also within 120 hours after the onset of the at least one symptom of ACS; (d) determining a second concentration of the at least one cardiac marker in the second blood sample collected at the second time point by performing an immunomagnetic reduction (IMR) assay; (e) comparing the first concentration of the at least one cardiac marker obtained in step (b) with the second concentration of the at least one cardiac marker obtained in step (d); and (f) administering a treatment for ACS to the subject whose first concentration of the at least one cardiac marker is higher or lower than the second concentration of the at least one cardiac marker.

As used herein, ACS covers any group of clinical symptoms compatible with acute myocardial ischemia. Acute myocardial ischemia is chest pain due to insufficient blood supply to the heart muscle that results from coronary artery disease (CAD), and includes acute myocardial infarction (AMI) and unstable angina pectoris (UAP).

When the subject has acute chest pain or angina equivalent symptoms, the method of the present invention can be used for diagnosing whether the subject suffers from ACS. In some embodiments, the method is useful for early diagnosis of three forms of ACS, including unstable angina pectoris (UAP), non-ST elevation myocardial infarction (NSTEMI), and ST-elevation myocardial infarction (STEMI).

The method of the present invention although can be used for any patient, at any time, they are particularly useful for those patients for whom a diagnosis or the severity of a condition associated with cardiac ischemia is difficult to determine. For example, a patient may have symptoms suggesting ACS, e.g., chest pain, nausea, and/or shortness of breath, but have normal or non-diagnostic electrocardiogram (ECG) results, or a level of a circulating biomarker, e.g., CPK, CK-MB, Tn-I/T, that is below what is considered as critical or diagnostic. For such patients, the method of the present invention can be used for early diagnosis and treatment of ACS. Therefore, the method of the present invention is used for a subject suspected of having ACS, i.e., the subject has at least one symptom of ACS. In some embodiments, the at least one symptom of ACS is chest pain, shortness of breath or a combination thereof.

The term “patient” or “subject” is used throughout the specification to describe an animal, e.g., a rodent or non-rodent, or a human, to whom diagnosis according to the methods of the present invention is provided. Veterinary and non-veterinary applications are contemplated. The term “patient” or “subject” includes, but is not limited to, a mammal, e.g., a human, other primates, a pig, a rodent such as a mouse or a rat, a rabbit, a guinea pig, a hamster, a cow, a horse, a cat, a dog, a sheep and a goat. Typical patients include humans, farm animals, horses, and domestic pets such as cats and dogs. In a preferred embodiment, the subject is a human. In a more preferred embodiment, the subject is a patient having at least one symptom of ACS.

As used herein, the term “blood sample” refers to a biological sample derived from blood, preferably peripheral (or circulating) blood. The blood sample can be whole blood, plasma or serum. In a preferred embodiment, the blood sample is serum.

In one embodiment, the first and second time points are within 96 hours after the onset of the at least one symptom of ACS in the subject. In one preferred embodiment, the first and second time points are within 72 hours after the onset of the at least one symptom of ACS in the subject. In a more preferred embodiment, the first and second time points are within 48 hours after the onset of the at least one symptom of ACS in the subject.

In another embodiment, the interval between the first time point and the second time point is at least one hour. In a preferred embodiment, the interval between the first time point and the second time point is at least 2, 3, 4, 5, 6, 7, or 8 hours.

In one embodiment, the first concentration of the at least one circulating biomarker is 5% higher or lower than the second concentration of the at least one biomarker. In a preferred embodiment, the first concentration of the at least one biomarker is 10% higher or lower than the second concentration of the at least one biomarker.

As used herein, the cardiac markers are the circulating biomarkers. The circulating biomarkers include cardiac enzymes which are useful in the method of the present invention and can be associated with myocardial damage or myocardial infarction. For example, determining the concentration of a circulating biomarker, e.g., in serum or plasma, of a subject can be useful for diagnosing myocardial infarction. These circulating cardiac enzymes are known in the art, and include, but are not limited to, troponin-I (Tn-I), troponin-T (Tn-T), creatine kinase-MB (CK-MB), aspartate transaminase (AST), lactate dehydrogenase (LDH), ischemia-modified albumin (INA), alanine transaminase (ALT), and myoglobin (MyG). Among these, the troponin markers are considered to be the most sensitive and specific markers for cardiac damages. CK-MB is a particular isoenzyme of creatine kinase, and its concentration is typically expressed as a CK-MB Index (ratio of CK-MB to total creatine kinase). Myoglobin is a heme protein found in cardiac muscle that has attracted considerable interest as an early marker of ACS. In some embodiments, the at least one cardiac marker comprises myoglobin, Tn-I/or T and CK-MB.

Some circulating biomarkers could reflect atherosclerotic plaque burden or plaque rupture. The present invention further demonstrates that the fatty acid-binding protein 4 (FABP4) is a new biomarker which can be used in the early diagnosis and risk stratification of patients with acute chest pain and suspected ACS. FABP4, also known as adipocyte fatty acid-binding protein (A-FABP), is a member of the FABP superfamily and is highly expressed in adipose tissue by means of adipocytes and macrophages. It is thought that FABP's roles include fatty acid uptake, transport, and metabolism. It has been proven that increased circulating FABP4 levels are associated with obesity, insulin resistance, type 2 diabetes, hypertension, and atherosclerosis. Some researches using animals or human carotid endarterectomy samples indicate the involvement of FABP4 in the progression of atherosclerosis and plaque rupture, and suggest it may have early diagnostic and therapeutic potential (Yeung D C et al., 2007; Peeters W et al., 2010; Holm S et al., 2011; Saksi J et al., 2011; Lee K et al., 2012). However, the present invention demonstrates that release of FABP4 is detected in circulation 1-6 hours after acute chest pain, peaks at 8-12 hours, and returns to normal within 24-30 hours, in STEMI patients after primary revascularization.

In the present invention, the levels or concentrations of these cardiac markers can be determined by the immunomagnetic reduction (IMR) assay on plasma or serum samples of the subject. Multiple samples from a subject at different time points can be analyzed to determine dynamic changes in the levels of the cardiac markers. For example, samples can be obtained from the subject at regular intervals, e.g., every 1-8 hours, in a period of, e.g., 120 hours.

When the subject has significant dynamic changes in the level of the at least one specific cardiac marker, the present invention further administers guideline recommended therapies to the subject. In one embodiment, the guideline recommended therapies or the treatment for the ACS comprises a reperfusion therapy and an effective amount of an ACS-treating drug. In a preferred embodiment, the reperfusion therapy is a thrombolytic therapy, a coronary angioplasty or a bypass surgery. In another embodiment, the ACS-treating drug comprises thrombolytics, nitroglycerin, antiplatelet drug, beta blocker, angiotensin-converting enzyme (ACE) inhibitor, angiotensin receptor blocker (ARB) and statin.

As used herein, the level of the cardiac marker is a concentration of a specific biomarker. Usually, the unit of the concentration is pg/ml.

In some embodiments, the immunomagnetic reduction (IMR) assay that determines the first and the second concentration of the at least one cardiac marker in the steps (b) and (d) of the present method comprises: (i) detecting in vitro an IMR signal of the at least one biomarker in the blood sample, wherein the IMR signal is produced by the at least one cardiac marker bound with magnetic nanoparticles containing an anti-specific cardiac marker antibody; and (ii) calculating the first or the second concentration ϕ of the at least one cardiac marker in the blood sample by fitting the IMR signal of the at least one cardiac marker detected in step (i) to a logistic function (I):

$$IMR ( \% ) = \frac{A - B}{1 + {( \frac{φ}{{φ}_{o}} )}^{γ}} + B \qquad ( I )$$

wherein IMR (%) is the IMR signal of the at least one cardiac marker in a percentage value, fitting parameter A is a background value, B is a maximum value, ϕₒ is the concentration of the at least one cardiac marker when the IMR signal equals ((A+B)/2), and γ is a slope at data point ϕₒ of a curve where ϕ is x-axis and IMR (%) is y-axis.

As used herein, the formula (I) mentioned in the present invention refers to the formula used to convert IMR signals into concentrations and disclosed in the prior art (C. C. Yang et al., Effect of molecule-particle binding on the reduction in the mixed-frequency alternating current magnetic susceptibility of magnetic bio-reagents, Journal of Applied Physics, Volume 112, Issue 2, 2012). When using the formula (I), a graph is plotted where the concentration of the standard biomarker is the x-axis and the corresponding IMR signal is the y-axis; the background value (A) is then determined by detecting the IMR signal of a reagent free from a targeted biomarker; the maximum value (B) is determined by detecting the IMR signal of the reagent which is saturated with the targeted biomarker; a curve between points A and B is drawn on the graph, the concentration of the biomarker (ϕₒ) is a point on the curve where the IMR signal equals to ((A+B)/2); calculating the slope (γ) at ϕₒ; and the concentration of the to-be-detected biomarker (ϕ) can then be computed.

In one embodiment, the material of the magnetic nanoparticles is selected from the group consisting of Fe₃O₄, Fe₂O₃, MnFe₂O₄, CoFe₂O₄, and NiFe₂O₄. In a preferred embodiment, the material of the magnetic nanoparticles is Fe₃O₄.

In some embodiments, the anti-specific biomarker antibody comprises an anti-myoglobin antibody, an anti-troponin-I antibody, an anti-creatine kinase MB (CK-MB) antibody and an anti-FABP4 antibody.

In some embodiments, the method described herein further includes providing the levels of a specific biomarker, e.g., FABP4, in a subject from multiple time points, e.g., at least two, three, or more than three time points. Based on the value of the level of the biomarker at each time point, ACS in the subject is able to be diagnosed by calculating the dynamic curve of the level of a specific biomarker in a time course or comparing the difference in the level of a specific biomarker at different time points.

ACS is usually associated with coronary atherothrombosis, but can also be associated with cocaine use. Symptoms associated with ACS include, but are not limited to, chest pain, chest tightness, bradycardia, tachycardia, heart palpitations, dyspnea, shortness of breath, dizziness and cold sweating. The present invention is based, at least in part, on the discovery that the circulating level of a specific biomarker, FABP4, can be detected by using the IMR assay. FABP4 first appears 1-6 hours after onset of symptoms, peaks at 8-12 hours, and returns to normal within 24-30 hours, in STEMI patients after primary revascularization. Its release kinetics is helpful to early diagnosis of ACS. Thus, subjects presenting with one or more of these symptoms will benefit from the methods of the invention, as well as those subjects who have a family history of ACS or symptoms thereof, a genetic predisposition to ACS, other risk factors for ACS, and/or have previously suffered from ACS.

### EXAMPLES

The examples below are non-limiting and are merely representative of various aspects and features of the present invention.

Methods:

A total of six subjects who presented with a primary complaint of acute chest pain and who were suspected of having ACS in the emergency department were consecutively recruited into this invention. All enrolled patients were (1) aged 40-70 years of age, and consented to participate in the study group; (2) admitted to the emergency department due to chest pain or potential ACS. Serial ECG, cardiac enzymes including central lab CPK, CK-MB and troponin T (Tn-T) measurements in combination with the medical history and physical examination were evaluated. Coronary angiography and echocardiography were performed after admission. All patients were asked to provide a 10-ml non-fasting venous blood sample (K3 EDTA, lavender-top tube) in every 6-hour interval for 3 days. Each sample was anonymous to colleagues in the laboratory. The blood samples were centrifuged (2500 g for 15 minutes) within 1 hour of withdrawal, and plasma was aliquoted into cryotubes and stored at −20° C. IMR assessments of myoglobin (MyG), troponin-I (Tn-I), creatine kinase-MB (CK-MB) and fatty acid-binding protein 4 (FABP4) were performed to clarify the metabolic cycle of myoglobin, troponin I, CK-MB and FABP4.

Four kinds of reagents were used for assaying plasma myoglobin, troponin-I, CK-MB and FABP4, respectively. Each kind of reagent was composed of magnetic nanoparticles dispersed in a pH-7.2 phosphate buffer saline (PBS) solution. These reagents were prepared by immobilizing antibodies against myoglobin, troponin-I, CK-MB and FABP4 respectively on magnetic nanoparticles. Therefore, four kinds of antibodies were immobilized onto dextran coated Fe₃O₄ magnetic particles, and the antigens and antibodies used were tabulated in table 1.

TABLE 1
the antigens and antibodies of the present invention

| Antigen | Notation | Model |
|---|---|---|
| Human Myoglobin | MyG | ProSpec, pro-565 |
| Recombinant Human Cardiac | Tn-I | ProSpec, pro-324 |
| Troponin I | | |
| Human Creatine Kinase MB full | CK-MB | Abcam, |
| length protein | | ab109675 |
| FABP4 Antibody(2H3L2) Ab | FABP4 | Thermo Fisher |
| | | Scientific, |
| | | PIE701158 |
| Anti-Myoglobin antibody [4E2] | Anti-MyG | Abcam, |
| | | ab8343 |
| Anti-Cardiac Troponin I antibody | Anti-Tn-I | Abcam, |
| [EP1106Y] | | ab52862 |
| Anti-Creatine Kinase MB [CK1] | Anti-CK- | Abcam, ab404 |
| antibody | MB | |
| Adipocyte Fatty Acid Binding | Anti- | BioVendor, |
| Protein Human E. coli Tag free | FABP4 | RD172036100 |

Four kinds of reagents were used for assaying plasma myoglobin (MyG), troponin-I (Tn-I), CK-MB and FABP4, respectively. Each kind of reagent was composed of magnetic nanoparticles dispersed in a pH-7.2 PBS solution. These reagents were prepared by immobilizing antibodies against these cardiac enzymes respectively on magnetic nanoparticles. The mean diameter of antibody-functionalized magnetic nanoparticles was 50-60 nm. The magnetic concentrations of the MyG and CK-MB reagents were 5.0 mg-Fe/ml, while the magnetic concentration of the Tn-I and FABP4 reagent were 6.5 mg-Fe/ml.

60 μl of plasma was mixed with 40 μl of reagent at room temperature for the detection of immunomagnetic reduction signal, IMR (%). Each mixture was put into a SQUID-based alternative-current (ac) magnetosusceptometer (XacPro-E, MagQu) to detect the time dependent AC magnetic susceptibility. After antibody-functionalized magnetic nanoparticles had bound with target biomarkers, the AC magnetic susceptibility of the mixture reduced. The reduction in the magnetic susceptibility was referred as the IMR signal.

In this experiment, several solutions with various concentrations of the cardiac enzymes were prepared. These solutions were used as to-be-detected samples to establish the relationships between the IMR signal and the cardiac enzymes (e.g. myoglobin (MyG), troponin-I (Tn-I), CK-MB and FABP4), respectively. These relationships were referred to as characteristic curves. Then, IMR signals of human plasma for these cardiac enzymes were detected and converted to the concentrations of these cardiac markers by the characteristic curves.

The clinical information of 6 subjects was listed in Table 2.

TABLE 2
Clinical characteristics of study subjects.

| | | | | | | | | Peak | | | | | |
|---|---|---|---|---|---|---|---|---|---|---|---|---|---|
| | | | | Onset | ED to | Initial | Peak | CK- | Cath | | | | |
| | | | | time to | cath | Tn-T | CPK | MB | to peak | | | Cardiogenic | LVEF |
| Case | Sample | Age | Sex | ED (hr) | (hr) | (ng/ml) | (U/L) | (U/L) | time (hr) | IRA | Vessel(s) | shock | (%) |
| 1 | S10 | 43 | M | 0.82 | 2.55 | 8190 | 11977 | 602 | 2.62 | LAD | 1 | 1/VT, VF | 39 |
| 2 | S15 | 62 | F | 3.23 | 0.38 | 2701 | 1535 | 172 | 4.53 | RCA | 3 | 1/VT, VF, | 63 |
| | | | | | | | | | | | | AF, IABP | |
| 3 | S27 | 63 | M | 0.50 | 0.60 | 1362 | 1078 | 103 | 4.53 | LAD | 1 | 0 | 68 |
| 4 | S29 | 67 | F | 1.13 | 2.42 | 4163 | 2385 | 143 | 3.75 | LAD | 3 | 0 | 46 |
| 5 | S30 | 63 | M | 0.48 | 2.32 | 578 | 2699 | 125 | 6.62 | LAD | 1 | 0 | 68 |
| 6 | S31 | 57 | M | 2.78 | 0.92 | 1404 | 1317 | 104 | 11.72 | LAD | 2 | 0 | 62 |

M = male,
F = female,
ED = emergent department,
IRA = infarct related artery,
LAD = left anterior descending artery,
RCA = right coronary artery,
VT = ventricular tachycardia,
VF = ventricular fibrillation,
AF = atrial fibrillation,
IABP = intra-aortic balloon pump,
LVEF = left ventricular ejection fraction.
Tn-T, CPK, CK-MB from the central lab of the hospital.

Results:

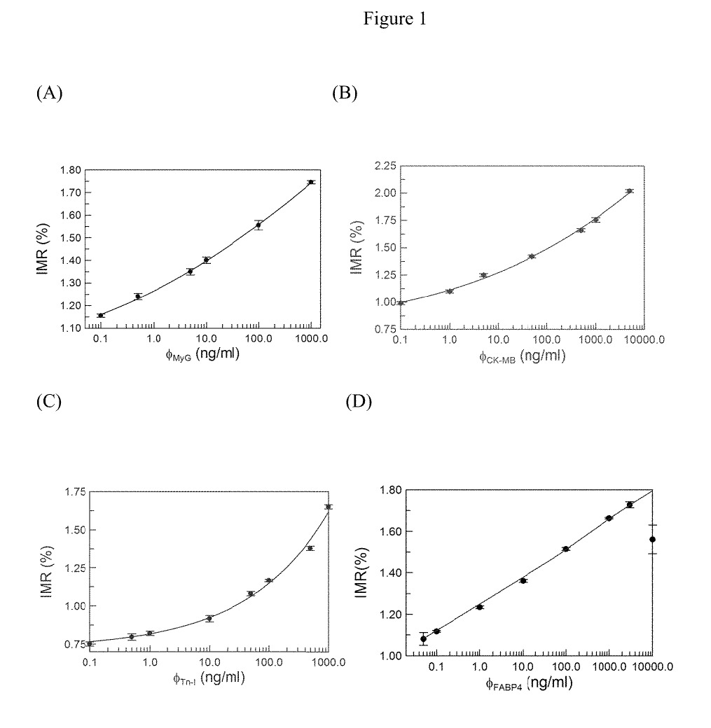

The concentration-dependent IMR signals, i.e. IMR (%)-ϕ curves or characteristic curves, for cardiac enzymes spiked in PBS were calculated to show with data points in FIG. 1(A)-(D). For a given cardiac enzyme, the data points were well fitted with the function (I)

$$IMR ( \% ) = \frac{A - B}{1 + {( \frac{φ}{{φ}_{o}} )}^{γ}} + B \qquad ( I )$$

wherein IMR (%) was the IMR signals of the cardiac enzyme, ϕ was the concentration of the cardiac enzyme, fitting parameter A was a background value, B was a maximum value, ϕₒ was the concentration of the cardiac enzyme when the IMR signal equals ((A+B)/2), γ was a slope at data point ϕₒ of a curve wherein ϕ was x-axis and IMR (%) was y-axis. The corresponding parameters for cardiac enzymes, listed in Table 2, were calculated by fitting data points to the function (I) (Chiu, M. J., “Combined plasma biomarkers for diagnosing mild cognition impairment and Alzheimer's disease”, ACS Chem. Neurosci. 4, 1530-1536 (2013)). The fitting curves in FIG. 1(A)-(D) were plotted in solid lines. The lowest detection limits for assays of myoglobin (MyG), troponin-I (Tn-I), CK-MB and FABP4 were down to a level of pg/ml.

TABLE 2
Fitting parameters in function (I) for the cardiac enzymes.

| Cardiac | Fitting parameter | | | |
|---|---|---|---|---|
| enzyme | A | B | ϕ₀ | γ |
| Myoglobin | 0.89 | 2.96 | 8544.8 | 0.167 |
| Troponin-I | 0.72 | 135.88 | 5.11 × 10⁹ | 0.423 |
| CK-MB | 0.73 | 5.50 | 2003527 | 0.169 |
| FABP4 | 0.781 | 2.235 | 92.89 | 0.178 |

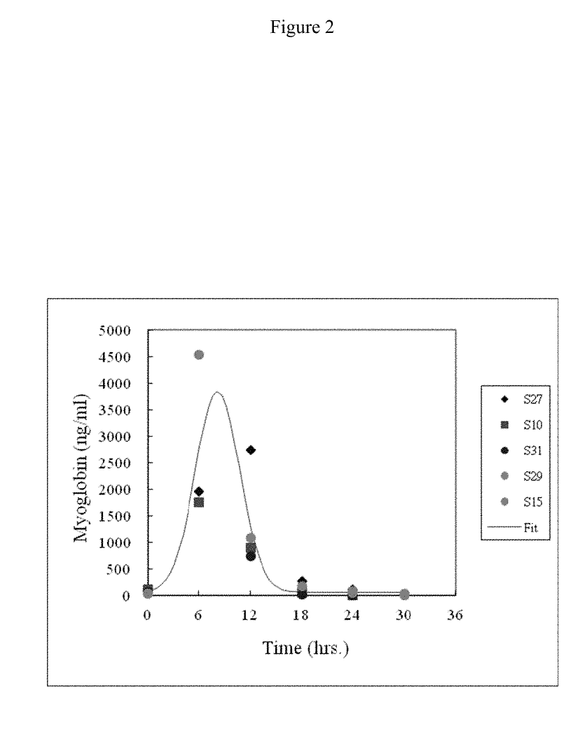

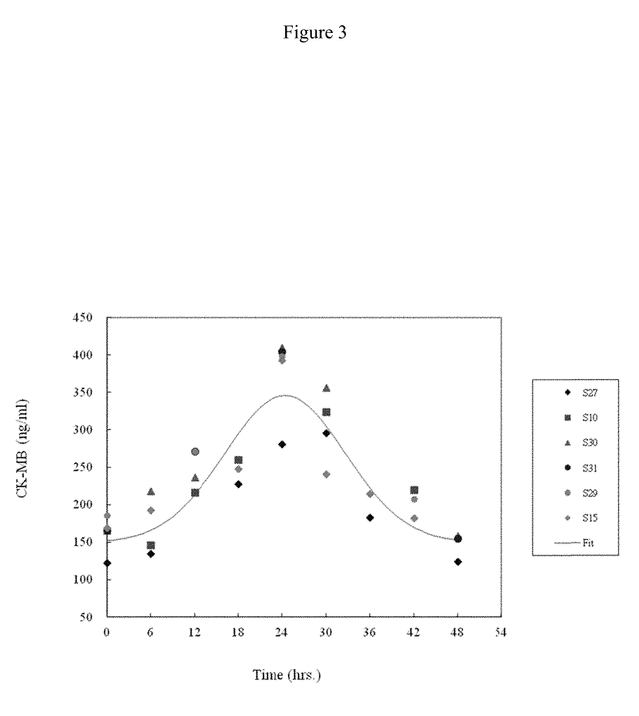

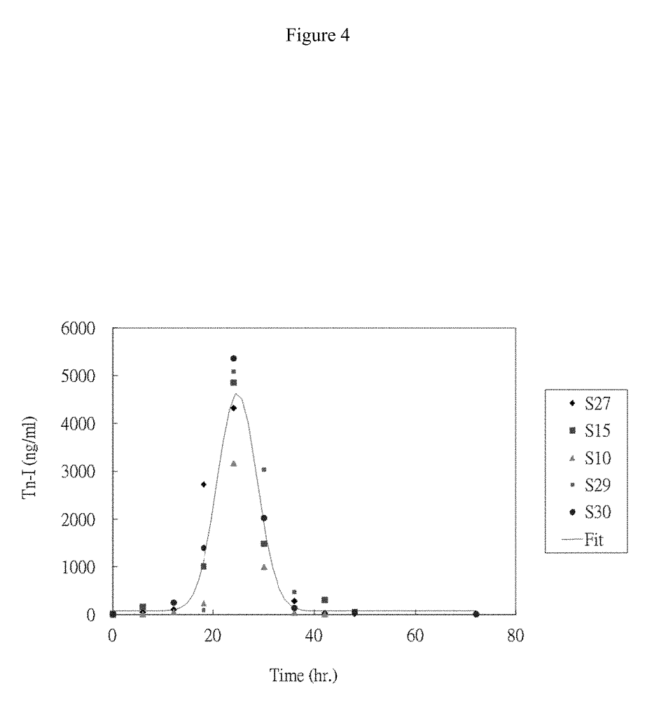

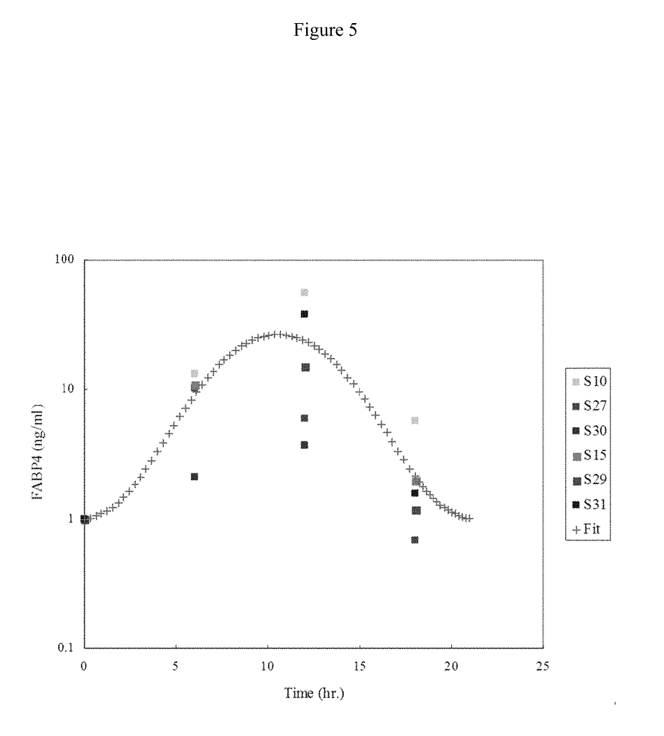

The present invention collected clinical data and IMR-detected concentration of myoglobin, CK-MB, troponin-I and FABP4 every 6 hours after onset of STEMI in 6 participants receiving primary coronary intervention. The present invention further established fitting curve of each cardiac enzyme based on the concentrations of the cardiac enzymes at each time point, respectively. FIGS. 2-5 showed the time activity curves of these cardiac enzymes (myoglobin (MyG), CK-MB, troponin-I (Tn-I), and FABP4). In FIG. 2, myoglobin rose 2-4 hours after onset of STEMI, peaked at 7-9 hours, and returned to normal within 24-36 hours. In FIG. 3, CK-MB first appeared 4-6 hours after onset of STEMI, peaked at 23-25 hours, and returned to normal in 45-48 hours. In FIG. 4, Tn-I first appeared 4-6 hours after onset of STEMI, peaked at 23-25 hours, and returned to normal in 40-48 hours. In FIG. 5, FABP4 rose 2-4 hours after onset of STEMI, peaked at 8-12 hours, and returned to normal within 22-25 hours. All subjects underwent primary coronary intervention, thus the peaking and duration could be delayed in patients without early coronary intervention.

In addition, the formula of the fitting curve for each cardiac enzyme was as follows:

$$\begin{matrix}Myoglobin \text{:} {φ}_{MyO} = 57.69 + \frac{24530.75}{5.16 \sqrt{π / 2}} exp ( \frac{- 2 {( t - 8.16 )}^{2}}{{5.16}^{2}} ) & ( 1 ) \\ CK \text{-} MB \text{:} {φ}_{CK - MB} = 149.6 + \frac{4051.5}{16.47 \sqrt{π / 2}} exp ( \frac{- 2 {( t - 24.40 )}^{2}}{{16.47}^{2}} ) & ( 2 ) \\ Tn \text{-} I \text{:} {φ}_{TrI} = 75.61 + \frac{45269.92}{7.74 \sqrt{π / 2}} exp ( \frac{- 2 {( t - 24.68 )}^{2}}{{7.74}^{2}} ) & ( 3 ) \\ FABP 4 \text{:} {φ}_{FABP 4} = 0.945 + \frac{193.81}{6.01 \sqrt{π / 2}} exp ( \frac{- 2 {( t - 10.54 )}^{2}}{{6.01}^{2}} ) & ( 4 )\end{matrix}$$

Fitting curves of IMR-detected cardiac enzyme as the function of time and peaking times were obtained. The peaking times of IMR-detected myoglobin, CK-MB, troponin-I and FABP4 were 8.2 hours, 24.4 hours, 24.7 and 10.5 hours, respectively.

The invention illustratively described herein suitably may be practiced in the absence of any element or elements, limitation or limitations, which are not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

What is claimed is:

1. A method for early diagnosing and treating acute coronary syndrome (ACS) in a subject in need thereof, the method comprising:
(a) providing a first blood sample collected at a first time point from the subject, wherein the first time point is within 120 hours after onset of at least one symptom of ACS in the subject;
(b) determining a first concentration of at least one cardiac marker in the first blood sample collected at the first time point by performing an immunomagnetic reduction (IMR) assay, wherein the at least one cardiac marker comprises a fatty acid-binding protein 4 (FABP4);
(c) providing a second blood sample collected at a second time point from the subject, wherein the second time point is also within 120 hours after the onset of the at least one symptom of ACS;
(d) determining a second concentration of the at least one cardiac marker in the second blood sample collected at the second time point by performing an immunomagnetic reduction (IMR) assay;
(e) comparing the first concentration of the at least one cardiac marker obtained in step (b) with the second concentration of the at least one cardiac marker obtained in step (d); and
(f) administering a treatment for ACS to the subject whose first concentration of the at least one cardiac marker is higher or lower than the second concentration of the at least one cardiac marker.

2. The method of claim 1, wherein the IMR assay that determines the first and the second concentration of the at least one cardiac marker in the steps (b) and (d) of claim 1 comprises:
(i) detecting in vitro an IMR signal of the at least one cardiac marker in the blood sample, wherein the IMR signal is produced by the at least one cardiac marker bound with magnetic nanoparticles containing an anti-specific cardiac marker antibody; and
(ii) calculating the first or the second concentration ϕ of the at least one cardiac marker in the blood sample by fitting the IMR signal of the at least one cardiac marker detected in step (i) to a logistic function (I):
$$IMR ( \% ) = \frac{A - B}{1 + {( \frac{φ}{{φ}_{o}} )}^{γ}} + B \qquad ( I )$$
wherein IMR (%) is the IMR signal of the at least one cardiac marker in a percentage value, fitting parameter A is a background value, B is a maximum value, φₒ is the concentration of the at least one cardiac marker when the IMR signal equals ((A+B)/2), and γ is a slope at data point ϕₒ of a curve where ϕ is x-axis and IMR (%) is y-axis.

3. The method of claim 1, wherein the ACS comprises unstable angina pectoris (UAP), non-ST elevation myocardial infarction (NSTEMI), and ST-elevation myocardial infarction (STEMI).

4. The method of claim 1, wherein the blood sample is serum.

5. The method of claim 1, wherein the at least one symptom of ACS is chest pain, shortness of breath or a combination thereof.

6. The method of claim 1, wherein the at least one cardiac marker comprises troponin-I (Tn-I), creatine kinase-MB (CK-MB) and myoglobin (MyG).

7. The method of claim 1, wherein the first and second time points are within 96 hours after the onset of the at least one symptom of ACS in the subject.

8. The method of claim 1, wherein the interval between the first time point and the second time point is at least one hour.

9. The method of claim 1, wherein the first concentration of the at least one cardiac marker is 5% higher or lower than the second concentration of the at least one cardiac marker.

10. The method of claim 2, wherein the material of the magnetic nanoparticles is selected from the group consisting of Fe₃O₄, Fe₂O₃, MnFe₂O₄, CoFe₂O₄ and NiFe₂O₄.

11. The method of claim 10, wherein the material of the magnetic nanoparticles is Fe₃O₄.

12. The method of claim 1, wherein the treatment for ACS comprises a reperfusion therapy or an effective amount of an ACS-treating drug.

13. The method of claim 12, wherein the reperfusion therapy is a thrombolytic therapy, a coronary angioplasty or a bypass surgery.
